(19) United States
(12) Patent Application Publication
(10) Pub. No.: US 2004/0057955 A1
(43) Pub. Date: Mar. 25, 2004

(54) Methods of inhibition of stenosis and/or sclerosis of the aortic valve

(76) Inventors: Kevin D. O'Brien, Seattle, WA (US); Catherine M. Otto, Seattle, WA (US); Jeffrey L. Probstfield, Kirkland, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/398,492

(22) PCT Filed: Oct. 5, 2001
(86) PCT No.: PCT/US01/31605

(51) Int. Cl. A61K 48/00; A61K 39/395; A61K 31/401; A61K 31/366

(52) U.S. Cl. 424/146.1; 514/44; 514/423; 514/460; 514/548

(57) ABSTRACT

The present invention provides methods for decreasing the amount and/or biological activity of angiotensin II in an aortic valve in an animal. The methods of the invention include administering to the animal an amount of an angiotensin converting enzyme antagonist and/or an angiotensin II type 1 receptor antagonist, effective to decrease the amount and/or biological activity of angiotensin II in the aortic valve in the animal.

### CROSS-REFERENCES TO RELATED APPLICATIONS

This application is based on prior U.S. Provisional Application Serial No. 60/238,367, the benefit of which is claimed under 35 U.S.C. § 119.

The invention described herein was supported, at least in part by National Institutes of Health, Grant No. DK-02345. The government has certain rights in the invention.

### FIELD OF THE INVENTION

The present invention relates to methods of treating calcific aortic valve disease of a mammal by the administration of agents capable of inhibiting the amount and/or activity of angiotensin II in the aortic valve of the mammal.

### BACKGROUND OF THE INVENTION

Calcific aortic valvular disease has been recognized as a distinct clinical entity since the early 1900's. Aortic valve changes are seen in 21-25% of adults over age 65 years, with a continuum of disease from aortic sclerosis to stenosis. Aortic sclerosis likely represents an early stage in the development of aortic stenosis, and refers to thickening and calcification of the aortic valve leaflets in the absence of obstruction to left ventricular outflow (Otto et al., N.Engl.J.Med.341:142-147; Stewart et al., J.Am.Coll.Cardiol.29:630-634). In aortic stenosis, the valvular pathology has progressed to cause obstruction to left ventricular outflow. Aortic stenosis, which has a prevalence of 2-3% in the elderly, is associated with a five-year risk of valve replacement or death of 80% (Ref). Recent studies have shown that the presence of aortic sclerosis on echocardiography, present in 20% of Americans over the age of 65 is associated with a 50% increased risk of cardiovascular mortality (Otto et al, N.Engl.J.Med.341:142-147).

No pharmacological therapy has been shown to decrease the progression of calcific aortic valvular disease. Thus, surgical replacement of the valve is currently the only treatment that can be offered to patients with this disease. Progress in the development of medical treatments for aortic sclerosis and stenosis have been hampered by the long-standing notion that calcific aortic valvular disease represents a degenerative condition that is an inevitable and unmodifiable consequence of aging. This notion has been challenged recently by a series of studies that have demonstrated that aortic valve disease is an active disease process. Recent studies have shown that aortic sclerosis and stenosis contain chronic inflammatory cells (Otto et al., Circulation 90:844-853; Olsson et al., J.Am.Coll.Cardiol.24:1664-1671; Olsson et al., J.Am.Coll.Cardiol.23:1162-1170), that a subset of aortic lesion cells actively express osteopontin, a molecule implicated in the regulation of calcification in both normal and pathological states (O'Brien et al., Circulation 92:2163-2168), and that plasma lipoproteins are deposited in aortic lesions (O'Brien et al., Arterioscler.Thromb.Vasc.Biol.16:523-532).

While many clinical factors associated with risk of developing atherosclerosis are shared by calcific aortic valvular disease, there is no previous literature that has even suggested the possibility of a role for the renin-angiotensin system in the pathogenesis of aortic sclerosis or stenosis. In fact, the use of pharmacological agents that inhibit angiotensin converting enzyme (ACE inhibitors) have traditionally been contra-indicated for the treatment of aortic valve disease, due to the presumed likelihood of adverse hemodynamic consequences (Swedberg et al, Eur. Heart J. 1996; 17:1306-11). Furthermore, the applicants are unaware of any previous literature suggesting that angiotensin converting enzyme is associated with plasma lipoproteins or lipoproteins found in aortic lesions.

### SUMMARY OF THE INVENTION

It has now been discovered that the renin-angiotensin system is involved in the progression of aortic valvular disease, and that angiotensin converting enzyme is associated with low density lipoproteins both in human plasma and in aortic lesions. Furthermore, it has been discovered that Angiotensin II type 1 (AT-1) receptors, which are the major cellular receptors for angiotensin II, are present in sclerotic through stenotic aortic valve lesions, but are not found in normal valve fibrosa. Accordingly, it has now been discovered that patients with aortic valve disease may be advantageously treated with pharmacological agents that decrease the amount and/or biological activity of angiotensin II in order to prevent the progression and/or complications of aortic valve disease.

In one aspect, the present invention provides new methods for treating a patient suffering from calcific aortic valve disease by administering to the patient a therapeutically effective amount of an agent capable of inhibiting the amount and/or activity of angiotensin II in the aortic valve of the patient.

In other aspects, the present invention provides methods for decreasing the amount and/or biological activity of angiotensin II in the aortic valve in an animal. In presently preferred, illustrative embodiments of this aspect of the invention, a human or non-human animal in need of such treatment is administered an amount of an agent, selected from the group consisting of angiotensin converting enzyme antagonists and angiotensin type 1 receptor antagonists, effective to decrease the amount and/or biological activity of angiotensin II in the aortic valve in the animal.

In yet other aspects, the present invention provides methods for decreasing the lipoprotein-mediated deposition of angiotensin converting enzyme in aortic valve lesions in an animal by decreasing the plasma lipoprotein levels in the animal and/or by inhibiting lipoproteins binding to aortic valve lesions, such as by inhibiting expression in the lesions of extracellular matrix molecules to which angiotensin converting enzyme-containing lipoproteins may bind.

The methods of the invention are useful in any situation where it is desirable to decrease angiotensin converting enzyme amount and/or biological activity. By way of example, the methods of the invention can be used to prevent the progression and/or complications associated with aortic valve disease.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 shows a photomicrograph with serial sections of a sclerotic human aortic valve with immunostaining for ACE (FIG. 1A), ApoB (FIG. 1B), and AngII (FIG. 1C) co-localization. In FIGS. 1A-1C, the aortic lumen is located at the top of the image and the left ventricular cavity is located at the bottom of the image. Positive immunohistochemical staining is indicated by a black reaction product.

FIG. 2 shows an immunoblot performed with a monoclonal antibody to ACE on LDL isolated from human plasma. A band of the expected molecular size of angiotensin converting enzyme (ACE) is detected on LDL. The location of molecular weight standards is labeled on the blot.

FIG. 3 shows a photornicrograph with positive immunohistochemical staining for angiotensin II Type 1 (AT-1) receptors on cells in a surgically-excised, stenotic aortic valve. Positive immunohistochernical staining is indicated by a black reaction product.

FIG. 4 graphically illustrates the results of a study using Electron Beam Tomography to quantify aortic valve calcium change in patients treated with statin compared with patients that did not receive statin.

### DETAILED DESCRIPTION OF THE PREFERRED EMBODIMENT

Unless specifically defined herein, all terms used herein have the same meaning as they would to one skilled in the art of the present invention. The following definitions are provided in order to provide clarity with respect to the terms as they are used in the specification and claims to describe the present invention.

As used herein, the term “pharmacological agent” refers to a substance other than food intended to affect the structure or function of a living body.

As used herein, the term “pharmaceutical compound” refers to a substance used as a medication according to the Food, Drug and Cosmetic Act.

As used herein, the term “ACE antagonist” and/or “ACE inhibitor” refer to a molecule, which decreases the amount or biological activity of angiotensin converting enzyme. ACE inhibitors are well known in the art for their activity in inhibiting ACE, thereby blocking conversion of the decapeptide angiotensin I to angiotensin II.

As used herein, the term “AT-1 antagonist” refers to a molecule, which decreases the amount or biological activity of angiotensin II type 1 receptor.

The term “low density lipoproteins” refers to an important class of serum lipoproteins in which a spherical hydrophobic core of triglycerides or cholesterol esters are surrounded by an amphipathic monolayer of phospholipids, cholesterol and/or apolipoproteins (especially apolipoproteins).

The term “aortic valve” refers to the heart valve that divides the left ventricle and the aorta. The aortic valve opens during left ventricular contraction and then closes to prohibit the backwash of oxygenated blood from the aorta into the ventricle. The aortic valve typically contains has 3 valve leaflets in most individuals, but may contain 2 valve leaflets in some individuals.

The term “aortic valve disease” refers to a disease state in which there is calcification and fibrosis of the aortic valve, encompassing aortic sclerosis and aortic stenosis.

The term “aortic sclerosis” refers to the thickening and calcification of the aortic valve leaflets in the absence of obstruction to left ventricular outflow.

The term “aortic stenosis” refers to a condition of valvular pathology in which left ventricular outflow is obstructed.

The term “statin” refers to HMG CoA reductase inhibitor.

The term “hemodynamic” refers to the dynamics of blood flow.

In one aspect, the present invention provides methods of decreasing the amount and/or biological activity of angiotensin II in an aortic valve in an animal. In this aspect of the invention, methods are provided for prophylactically and/or therapeutically treating an animal in need of such treatment, comprising administering to the animal an amount of an agent, selected from the group consisting of angiotensin converting enzyme (ACE) antagonists and angiotensin II type 1 receptor (AT-1) antagonists, effective to decrease the amount and/or biological activity of angiotensin II in the aortic valve in an animal. The methods described in the present invention are applicable to any animal, including mammals, such as human beings. The methods of this aspect of the invention can be used to decrease the amount and/or biological activity of angiotensin II in an aortic valve in any situation where such angiotensin II inhibition is desirable, including situations in which the animal is exhibiting aortic valve disease in the form of aortic sclerosis or aortic stenosis.

The methods of this aspect of the invention are useful, for example, to decrease or halt the progression of aortic sclerosis or aortic stenosis. In the context of this aspect of the method of the invention, aortic sclerosis refers to the thickening and calcification of the aortic valve leaflets in the absence of obstruction to the left ventricular outflow. This stage of the disease is characterized by a chronic inflammation with macrophage infiltrate, deposition of plasma lipoproteins, expression of osteopontin and extracellular mineralization. Aortic stenosis refers to the stage in aortic valvular disease in which the valvular pathology has progressed to cause obstruction to left ventricular outflow. This more severe disease state is characterized by disruption of normal valve architecture, severe calcification and macroscopic leaflet thickening.

A decrease in disease progression of aortic valvular disease is characterized by at least one of the following changes in a component of valvular disease pathology associated with aortic valve disease that occurs as a result of treatment in accordance with the methods of the invention: a decrease in the presence of an inflammatory cell infiltrate, a decrease in plasma lipoprotein deposition, a decrease in calcification of the leaflet (measured, for example, by imaging techniques); a decrease in the level of ACE, AngII or AT-1 in aortic valvular tissue (measured, for example, by immunohistochemistry); or a decrease in the rate of increase in blood flow velocity across the aortic valve, a decrease in transvalvular pressure gradient or a reduction in the rate of decrease of aortic valve function.

Imaging techniques useful in the practice of this aspect of the method of the invention include, but are not limited to: echocardiography, fluoroscopy and electron beam tomography (EBT). EBT is an especially useful imaging technique for detecting and quantifying changes in aortic valve leaflet calcification over time.

A decrease in the progression of aortic valvular disease may be characterized, for example, by a decrease in the percentage of patients being treated in accordance with the method of the invention that require valve replacement therapy or surgical intervention due to aortic valve disease. Other measurements useful to measure the progression of aortic valvular disease include functional tests such as pulmonary exercise tests.

In one embodiment of the methods of this aspect of the invention, the amount and/or biological activity of angiotensin II (AngII) is decreased in an animal by a method comprising administering to the animal an amount of an angiotensin converting enzyme (ACE) antagonist effective to decrease the amount of angeiotensin II in the aortic valve in the animal. In the practice of this aspect of the invention, representative ACE antagonists include: pharmaceutical compounds that inhibit the amount and/or biological activity of ACE (ACE inhibitors), ACE antisense nucleic acid molecules (such as antisense MRNA, antisense DNA or antisense oligonucleotides), ACE ribozymes, molecules that inhibit the biological activity of ACE (such as anti-ACE antibodies, or a blocking peptide which interacts with the active site of ACE) and molecules that decrease the amount of ACE carried into the aortic valve by plasma low density lipoproteins (LDLs). The methods of this aspect of the invention can be used to prevent the progression and/or decrease the risk of clinical events associated with aortic valve disease.

Any pharmaceutical agent that inhibits ACE and is effective to prevent the progression and/or complications associated with aortic valve disease may be used as an ACE antagonist or inhibitor in the practice of the present invention. Representative ACE inhibitors for use in the practice of the invention include, without limitation, alacepril, alatriopril, altiopril calcium, ancovenin, benazepril, benazepril hydrochloride, benazeprilat, benzazepril, benzoylcaptopril, captopril, captopril-cysteine, captopril-glutathione, ceranapril, ceranopril, ceronapril, cilazapril, cilazaprilat, converstatin, delapril, delapril-diacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat. Suitable ACE inhibitors to be employed in the practice of the invention are well known in the art, and several are used routinely for treating hypertension. For example, captopril and its analogs are described in U.S. Pat. Nos. 5,238,924 and 4,258,027. Enalapril, enalaprilat, and closely related analogs are described in U.S. Pat. Nos. 4,374,829, 4,472,380, and 4,264,611. Moexipril, quinapril, quinaprilat, and related analogs are described in U.S. Pat. Nos. 4,743,450 and 4,344,949. Ramipril and its analogs are described in U.S. Pat. Nos. 4,587,258 and 5,061,722. All of the foregoing parents are incorporated herein by reference for their teaching of typical ACE inhibitors that can be utilized according to this invention. Presently preferred ACE inhibitors that are approved for use in humans and are commercially available include, without limitation, ramipril, quinapril, captopril, lisinopril, benazepril, enalapril and fosinopril.

Effective amounts of ACE inhibitors for use in the practice of the invention will vary depending on the nature of the inhibitor, but will generally be within the amounts generally employed in the art for administration of the inhibitor for other purposes. For example, the ACE inhibitor may generally be administered at dosages in the range of about 0.05 to about 500 mg/day, more preferably about 0.1 to about 250 mg/day and most preferably about 0.2 to about 100 mg/day. Optimum dosage will be readily apparent to those skilled in the art for a particular inhibitor. The ACE inhibitors will generally be administered to a patient for a period of time sufficient to effect a measurable inhibition of the progression of aortic sclerosis and/or stenosis, as described herein.

ACE antisense nucleic acid molecules useful as ACE antagonists in the practice of the invention may be constructed in a number of different ways provided they are capable of interfering with the expression ACE. For example, an antisense nucleic acid molecule can be constructed by inverting the coding region (or a portion thereof) of ACE relative to its normal orientation for transcription to allow the transcription of its complement.

The antisense nucleic acid molecule is usually substantially identical to at least a portion of the target gene or genes. The nucleic acid, however, need not be perfectly identical to inhibit expression. Generally, higher homology can be used to compensate for the use of a shorter antisense nucleic acid molecule. The minimal percent identity is typically greater than about 65%, but a higher percent identity may exert a more effective repression of expression of the endogenous sequence. Substantially greater percent identity of more than about 80% typically is preferred, though about 95% to absolute identity is typically most preferred.

The antisense nucleic acid molecule need not have the same intron or exon pattern as the target gene, and non-coding segments of the target gene may be equally effective in achieving antisense suppression of target gene expression as coding segments. A DNA sequence of at least about 30 or 40 nucleotides may be used as the antisense nucleic acid molecule, although a longer sequence is preferable. In the present invention, a representative example of a useful antagonist of ACE is an antisense ACE nucleic acid molecule which is at least ninety percent identical to the complement of the ACE cDNA consisting of the nucleic acid sequence set forth in SEQ ID NO: 1. The nucleic acid sequence set forth in SEQ ID NO: 1 encodes the ACE protein consisting of the amino acid sequence set forth in SEQ ID NO: 2.

The targeting of antisense oligonucleotides to bind ACE mRNA is another mechanism that may be used to reduce the level of ACE protein synthesis. For example the synthesis of polygalacturonase and the muscarine type 2 acetylcholine receptor are inhibited by antisense oligonucleotides directed to their respective mRNA sequences (U.S. Pat. No. 5,739,119 and U.S. Pat. No. 5,759,829). Furthermore, examples of antisense inhibition have been demonstrated with the nuclear protein cyclin, the multiple drug resistance gene (MDG1), ICAM-1, E-selectin, STK-1, striatal GABA_{A} receptor and human EGF (see, e.g., U.S. Pat. No. 5,801,154; U.S. Pat. No. 5,789,573; U.S. Pat. No. 5,718,709 and U.S. Pat. No. 5,610,288).

In another embodiment of this aspect of the present invention, the ACE antagonist is an anti-ACE antibody. By way of representative example, antigen useful for raising antibodies can be prepared in the following manner. A nucleic acid molecule (such as an ACE cDNA molecule) is cloned into a plasmid vector, such as a Bluescript plasmid (available from Stratagene, Inc., La Jolla, Calif.). The recombinant vector is then introduced into an E. coli strain (such as E. coli XL1-Blue, also available from Stratagene, Inc.) and the polypeptide encoded by the nucleic acid molecule is expressed in E. coli and then purified. Alternatively, polypeptides can be prepared using peptide synthesis methods that are well known in the art. The synthetic polypeptides can then be used to prepare antibodies. Direct peptide synthesis using solid-phase techniques (Stewart et al., Solid-Phase Peptide Synthesis, W H Freeman Co, San Francisco Calif. (1969); Merrifield, J. Am. Chem. Soc. 85:2149-2154 (1963) is an alternative to recombinant or chimeric peptide production. Automated synthesis may be achieved, for example, using Applied Biosystems 431A Peptide Synthesizer (Foster City, Calif.) in accordance with the instructions provided by the manufacturer. Methods for preparing monoclonal and polyclonal antibodies are well known to those of ordinary skill in the art and are set forth, for example, in chapters five and six of Antibodies A Laboratory Manual, E. Harlow and D. Lane, Cold Spring Harbor Laboratory (1988). Antibody production includes not only the stimulation of an immune response by injection into animals, but also analogous processes such as the production of synthetic antibodies, the screening of recombinant immunoglobulin libraries for specific-binding molecules (Orlandi et al., Proc. Natl. Acad. Sci. USA 86:3833, 1989, or Huse et al. Science 256:1275, 1989), or the in vitro stimulation of lymphocyte populations.

The invention also extends to non-antibody polypeptides, sometimes referred to as blocking peptides, that have been designed to bind specifically to, and inhibit the active site of ACE. For example, the domain of ACE, which binds to the substrate angiotensin I can be targeted with a blocking peptide. Other examples of the design of such peptides, which possess a prescribed ligand specificity, are given in Beste et al. (1999, Proceedings of the National Academy of Science 96:1898-1903).

An additional strategy suitable for suppression of target gene activity entails the sense expression of a mutated or partially deleted form of the protein encoded by the target gene according to general criteria for the production of dominant negative mutations (Herskowitz I, Nature 329: 219-222 (1987)).

Ribozymes can also be utilized to decrease the amount and/or biological activity of ACE, such as ribozymes, which target ACE mRNA. Ribozymes are catalytic RNA molecules that can cleave nucleic acid molecules having a sequence that is completely or partially homologous to the sequence of the ribozyme. In this aspect of the invention, ribozyme transgenes are designed that encode RNA ribozymes that specifically pair with a target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the antisense constructs.

Ribozymes useful in the practice of the invention typically comprise a hybridizing region, of at least about nine nucleotides, which is complementary in nucleotide sequence to at least part of the target ACE mRNA, and a catalytic region which is adapted to cleave the target ACE MRNA (see generally, EPA No. 0 321 201; WO88/04300; Haseloff & Gerlach, Nature 334:585-591 [1988]; Fedor & Uhlenbeck, Proc. Natl. Acad. Sci.: USA 87:1668-1672 [1990]; Cech & Bass, Ann. Rev. Biochem. 55:599-629 [1986]).

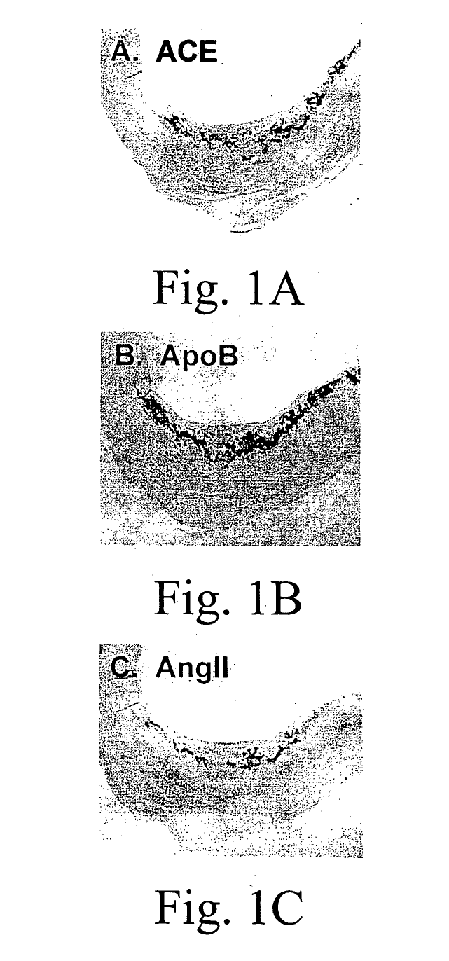

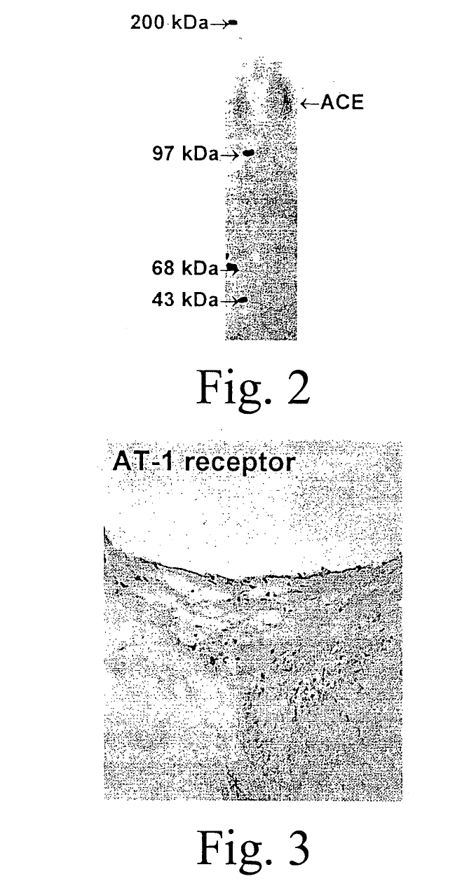

The present invention is based at least in part on the discovery that ACE is found in aortic lesions and is associated with low density lipoproteins such as apolipoprotein B as shown in FIG. 1. Furthermore, it has been found that ACE is associated with LDLs in human plasma as shown in FIG. 2. In accordance with these findings, any pharmaceutically acceptable agent that effectively decreases the amount and/or biological activity of ACE in an aortic valve, including a decrease in ACE that is carried into the aortic valve in association with low density lipoprotein(s) (LDLs), may be used as an ACE antagonist in the context of this aspect of the method of the invention. ACE antagonists useful in the practice of this aspect of the invention may either reduce the level of LDL in plasma, therefore limiting the availability of LDL with which ACE could associate, or the molecule may interfere with the binding and/or retention of ACE-containing LDL particles in an aortic valve lesion. Examples of molecules that reduce the level of LDL in plasma include statin, lovastatin, pravastatin, simvastatin, atorvastatin, rozuvastatin, nicotinic acid, bile acid resins such as colestipol and cholestyramine, fibric acid derivatives such as gemfibrozil, bezafibrate and fenofibrate, and cholesterol adsorption inhibitors such as ezetemibe.

In another embodiment of this aspect of the method of the invention, an ACE antagonist may be a molecule that interferes with association of ACE and LDL particles carried in plasma. Examples of molecules useful in this aspect of the invention include, but are not limited to, blocking antibodies and small peptides.

In another embodiment of this aspect of the invention, the biological activity of angiotensin II (AngII) is decreased in an animal by a method comprising the step of introducing into the animal an amount of an angiotensin type 1 receptor (AT-1) antagonist effective to decrease the biological activity of angiotensin II in the aortic valve in the animal. This aspect of the invention is based on the finding that Angiotensin II type 1 (AT-1) receptors which are the major cellular receptors for angiotensin II, are present in sclerotic through stenotic aortic valve lesions (as shown in FIG. 3), but are not found in normal valve fibrosa. Therefore, useful AT-1 antagonists in the practice of this aspect of the invention include: pharmaceutical compounds that inhibit the amount and/or biological activity of AT-1, AT-1 antisense nucleic acid molecules (such as antisense RNA, antisense DNA or antisense oligonucleotides), AT-1 ribozymes, or blocking peptides that interact with the extracellular domain of AT-1.

Pharmaceutical compounds that are useful in the practice of this aspect of the method of the invention include any compounds that inhibit or block angiotensin type 1 receptors and prevent the progression and/or decrease the risk of clinical events associated with aortic valve disease. Examples of angiotensin receptor blocking compounds that are approved for use in humans and are commercially available include: losartan, valsartan, irbesatan, telmesartan and candesartan.

In the practice of this aspect of the present invention, other representative examples of useful antagonists of AT-1 are antisense AT-1 nucleic acid molecules which are at least ninety percent identical to the complement of the AT-1 cDNA consisting of the nucleic acid sequence set forth in SEQ ID NO: 3. The nucleic acid sequence set forth in SEQ ID NO: 3 encodes the AT-1 protein consisting of the amino acid sequence set forth in SEQ ID NO4. Representative methods of constructing an AT-1 antisense nucleic acid molecule include any methods of constructing antisense nucleic acid molecules described in this patent application.

In another embodiment of this aspect of the invention, the AT-1 antagonist may be an anti-AT-1 antibody. Representative methods for the preparation of useful antibodies include any methods of antibody preparation known in the art and/or described in this patent application, including the use of recombinant protein and peptide synthesis. The invention also extends to blocking peptides that have been designed to specifically bind and inhibit the angiotensin type 1 receptor substrate binding domain. Other examples of useful blocking peptides include peptides that inhibit binding of LDL to the extracellular matrix molecules that are present in aortic lesions.

Ribozymes useful in the practice of the invention comprise a hybridizing region of at least nine nucleotides, which is complementary in nucleotide sequence to at least part of the target AT-1 MRNA, and a catalytic region which is adapted to cleave the target AT-1 mRNA. Representative methods of producing an AT-1 ribozyme include any methods of ribozyme preparation described in this patent application.

Molecules that decrease the amount and/or biological activity of angiotensin II, including ACE antagonists and AT-1 antagonists, can be delivered into the body of an animal by any suitable means. By way of representative example, said antagonists can be introduced into an animal body by application to a bodily membrane capable of absorbing the composition, for example the nasal, gastrointestinal and rectal membranes. For transdermal applications, said antagonist molecules may be combined with other suitable ingredients, such as carriers and/or adjuvants. There are no limitations on the nature of such other ingredients, except that they must be pharmaceutically acceptable and efficacious for their intended administration, and cannot degrade the activity to the active ingredients of the composition. Examples of suitable vehicles include ointments, creams, gels or suspensions. ACE and AT-1 antagonist molecules also may be impregnated into transdermal patches, plasters and bandages, preferably in liquid or semi-liquid form.

Methods of delivery of ACE and AT-1 antagonist molecules also include the administration by oral, pulmonary, parenteral (e.g., intramuscular, intraperitoneal, intravenous (IV) or subcutaneous injection), inhalation (such as a fine powder formulation), transdermal, nasal, vaginal, rectal, or sublingual routes of administration, and can be formulated in dosage forms appropriate for each route of administration.

ACE and AT-1 antagonist molecules that are susceptible to degradation, such as blocking peptides, may be introduced in association with another molecule, such as a lipid, to protect the peptide from enzymatic degradation. For example, the covalent attachment of polymers, especially polyethylene glycol (PEG), has been used to protect certain proteins and peptides from enzymatic hydrolysis in the body and thus prolong half-life (F. Fuertges, et al., J. Controlled Release, 11: 139 (1990)).

Antisense ACE or AT-1 nucleic acid molecules can be delivered by any art recognized method of delivering nucleic acid molecules into living cells including transduction, transfection, transformation, direct injection, electroporation, virus-mediated gene delivery, amino acid-mediated gene delivery, biolistic gene delivery, lipofection and heat shock. See, generally, Sambrook et al, supra. Representative, non-viral, methods of gene delivery into cells are disclosed in Huang, L., Hung, M-C, and Wagner, E., Non-Viral Vectors for Gene Therapy, Academic Press, San Diego, Calif. (1999).

While the preferred embodiment of the invention has been illustrated and described, it will be appreciated that various changes can be made therein without departing from the spirit and scope of the invention.

### EXAMPLE 1

This' example shows that human aortic valve lesions contain angiotensin converting enzyme (ACE), which co-localizes with angiotensin II and apolipoprotein B.

Preparation of human aortic valve tissue: Aortic valves studied were obtained from a total of 26 adults obtained either at autopsy (n=17), surgery (n=4) or from hearts explanted at the time of cardiac transplantation (n=5). Valve tissues from explanted hearts were fixed in 10% neutral buffered formalin within 2 hours of organ removal. Valve tissues obtained from autopsy or surgery were fixed in methanol-Carnoy's solution (60% methanol/30% chloroform/10% glacial acetic acid). After fixation, specimens were embedded in paraffin wax.

Histological grading of aortic lesion severity: Paraffin-embedded specimens were examined for morphologic and cellular features using hematoxylin, eosin and Movat's stains. Aortic valvular lesions were defined morphologically as focal areas of mononuclear inflammatory cell infiltrates and extracellular matrix expansion involving the subendothelial region on the aortic side of the leaflets extending into the fibrosa layer of the valve. Histological grading of lesion severity was designated as follows. Early lesions were characterized by subendothelial thickening on the aortic side of the leaflets in areas of disruption of the basement membrane, displacement of the elastic lamina and accumulation of lipid, protein, extracellular mineralization and cellular infiltrate. Late lesions were characterized by significant leaflet destruction with disruption of normal leaflet architecture, severe calcification and macroscopic thickening of the leaflets.

Immunohistochemistry methods: Immunohistochemical studies were performed using the following, polyclonal antibodies: 1) a sheep polyclonal antibody recognizing angiotensin converting enzyme (ACE) (titer 1:750, overnight, Chemicon International, Inc., Temecula, Calif.), 2) a rabbit polyclonal antibody recognizing angiotensin II (AngII)(titer 1:50, overnight, Cortex Biochem., San Leandro, Calif.) and 3) a rabbit polyclonal antibody recognizing human apolipoprotein B (titer 1:1000, 60 minute incubation, kind gift of Dr. Thomas Innerarity, Gladstone Institute, San Francisco, Calif.). The anti-apoB antiserum was extensively characterized and shown to be monospecific for human apoB (data not shown).

In order to perform immunohistochemical analysis, six micrometer sections of human aortic valve tissue were placed on glass microscope slides, deparaffinized and then endogenous peroxidase activity was blocked with 3% H₂O₂ (Sigma Corp., St. Louis, Mo.). Specimens were washed with phosphate-buffered saline (PBS), and then incubated for either 60 minutes or overnight with the primary antibody. The specimens were washed again with PBS and a biotin-labeled anti-rabbit or anti-sheep antibody was then applied for 30 minutes. An avidin-biotin-peroxidase conjugate (ABC Elite, Vector Laboratories, Burlingame, Calif.) was then applied for 30 minutes. Standard peroxidase enzyme substrate, 3,3′-diaminobenzidine (Sigma) with NiCl₂ was added to yield a black reaction product. Cell nuclei were counterstained with methyl green.

Results of Immunohistochemical Analysis: In each specimen, the three anatomic layers (fibrosa, spongiosa and ventricularis) of the valve leaflets were identified and the specimen was characterized as an early or late lesion as previously described. All of the tissue specimens evaluated contained portions of the aorta and several specimens also contained an adjacent coronary artery. The positive ACE staining within these structures served as an internal positive control for the anti-ACE antibody. The ACE staining was localized to the intima of the aorta in all 26 specimens. Microvessels within the adventia of adjacent coronary arteries and of the aorta also stained positively for ACE.

In normal valves, ACE was detected only on endothelial cells (data not shown). However, in aortic valve lesions ranging from sclerotic to severely stenotic, ACE was detected in two distributions. The first distribution was cellular, with ACE staining present in macrophages located within lesions. In contrast, resident macrophages within the spongiosa layer did not contain ACE, suggesting that ACE expression was upregulated in lesion macrophages. The second distribution of ACE was extracellular, with ACE being detected only in regions that also contained apolipoprotein B (see FIG. 1). Of the two ACE distributions, the extracellular pattern was much more widespread. In addition, the enzymatic product of ACE, AngII, was detected in regions of the lesions that contained extracellular ACE and ApoB, indicating that the extracellular ACE was enzymatically active (see FIG. 1).

The discovery that ACE is present and enzymatically active in aortic lesions demonstrates the utility of treating patients exhibiting aortic valve sclerosis or stenosis with pharmacological inhibitors of the angiotensin converting enzyme to prevent progression and complications of aortic valve disease.

### EXAMPLE 2

This example shows that AngII Type 1 (AT-1) receptors were detected in human aortic valve lesions and were not detected in normal valve fibrosa. Furthermore, the AT-1 receptors were more prominent in late lesions, suggesting up-regulation of these receptors with increasing disease severity.

Immunohistochemistry methods: Immunohistochemistry was performed with a rabbit polyclonal antibody to the angiotensin II type 1 receptor (AT-1, titer 1:200, overnight, Santa Cruz Biotechnology, Inc.) on the aortic valve tissues described in Example 1. In addition, an antibody was used against alpha smooth muscle actin (anti-α-actin, titer 1:1000 for one hour, Boehringer-Mannheim Biochemica), which is expressed by myofibroblasts.

Results of Immunohistochemical Analysis: AT-1 receptors were not detected in fibroblasts of normal valve fibrosa (data not shown). However, AT-1 receptors were detected in sclerotic through stenotic aortic valve lesions (see FIG. 3). Furthermore, AT-1 receptors were more prominent in late lesions, suggesting up-regulation of these receptors with increasing disease severity. AT-1 receptors, when present in aortic valve lesions, were localized to a subset of α-actin positive fibroblasts (myofibroblasts). These myofibroblasts were present in increased numbers at the leaflet tips of both normal and lesion-containing valves.

The discovery that AT-1, the major cellular receptor for AngII is expressed in aortic lesions demonstrates the utility of treating patients exhibiting aortic valve sclerosis or stenosis with pharmacological inhibitors of the angiotensin II Type 1 receptor to prevent progression and complications of aortic valve disease.

### EXAMPLE 3

This examples shows that ACE is present on human plasma lipoproteins.

Lipoprotein isolation: Low density lipoprotein (LDL, d 1.019 to 1.063) was isolated by sequential density-gradient ultracentrifugation, or by fast protein liquid chromatography from plasma obtained from a pool of 6 normal human volunteers. LDL was dialyzed extensively at 4° C. in the dark against 150 mmol/L NaCl and 1 mmol/L EDTA (pH 7.40) before use in immunoblot analysis.

Immunoblot Analysis: LDL samples were electrophoresed on 4-12% polyacrylamide gels under reducing conditions and then transformed to nitrocellulose membranes using a Mini Trans-Blot Cell (Bio-Rad Laboratories, Hercules, Calif.). Angiotensin converting enzyme was detected using either the sheep polyclonal antibody (anti-ACE, titer 1:1000 or 1:570, Chemicon International, Inc., Temecula, Calif.) or a mouse monoclonal antibody (anti-ACE, titer 1:570, Chemicon International, Inc., Temecula, Calif.), followed by the appropriate (anti-sheep or anti-mouse) secondary antibodies and enhanced chemiluminescence (Western Light Chemiluminescent Detection System with CSPD substrate, Tropix, Bedford, Mass.).

Results of Immunoblot Analysis: Using the polyclonal antibody to ACE, the LDL fractions isolated by either ultracentrifugation or FPLC both were found to contain the ACE protein. (see FIG. 2). In addition, Western blotting performed with a commercially-available monoclonal antibody to ACE also confirmed the presence of ACE in both ultracentrifugally or FPLC isolated LDL fractions. Both antibodies to ACE detected on LDL a single band of ≅150-160 kiloDaltons molecular size, demonstrating that ACE is carried on LDL in plasma.

The discovery that ACE is present in association with LDL in human plasma as well as in aortic lesions demonstrates the utility of treating patients with aortic valve sclerosis or stenosis with pharmacological agents that decrease the delivery of ACE by LDL to lesions, either by a) lowering plasma LDL levels or b) interfering with LDL retention on lesion extracellular matrix. The applications of a therapy that decreases the amount of ACE associated with plasma LDL extends beyond aortic valve disease to a variety of disease states including atherosclerosis, renal diseases, pulmonary diseases, hepatic diseases, reproductive diseases and neurological diseases where lipoproteins serve as a vehicle for delivery of ACE to tissues.

### EXAMPLE 4

This example demonstrates that no adverse hemodynamic effects were seen with short-term use of an ACE inhibitor in patients with mild to moderate aortic stenosis.

Clinical Protocol: This pilot study enrolled 8 patients with mild to moderate aortic stenosis, normal left ventricular (LV) function and no history of coronary artery disease. At baseline, patients had an echocardiogram, history, measurements of blood pressure and heart rate and a basic chemistry panel. Patients then were placed on the ACE inhibitor ramipril, 2.5 mg qd for 2 weeks followed by up-titration at 2 week intervals to 2.5 mg bid, 5 mg bid and 7.5 mg bid. The study lasted 8 weeks. Vital signs were repeated weekly, chemistry panels every 2 weeks and echocardiograms every 4 weeks.

Results: There were no significant differences between baseline (see Table 1) and final measurements (mean±SD) for sitting heart rate (66±9 vs. 62±3), systolic BP (118±21 vs. 114±14 mmHg) or diastolic BP (68±7 vs. 65±10 mmHg) (see Table 2), potassium (4.4±0.5 vs. 4.3±0.5 meq/dL), creatinine (1.1±0.3 vs. 1.1±0.3 mg/dL) or glucose (105±46 vs. 91±18 mg/dL) (see Table 3). Also, there were no significant differences between baseline and final echocardiographic measurements of aortic stenosis jet velocity (2.8±0.2 vs. 2.8±0.3 m/sec), left ventricular outflow tract velocity (1.0±0.2 vs. 1.0±0.2 m/sec), cardiac output (5.2±1.1 vs. 5.7±1.4 L/min) or LV ejection fraction (72±4 vs. 71±7%) (see Table 4).

TABLE 1
Baseline Characteristics

| | N = 8 |
|---|---|
| Age, mean ± SD | 60 ± 19 |
| Male gender, % | 88% |
| Hypertension, % | 17% |
| Smoking, % | 17% |
| Hypercholesterolemia, % | 33% |
| Diabetes, % | 17% |
| B-blockers, % | 80% |
| Ca channel blockers, % | 17% |
| Diuretic, % | 17% |
| AS-Jet (m/s), mean ± SD | 2.8 ± 0.2 |
| Potassium (mEq/L), mean ± SD | 4.4 ± 0.5 |
| Creatinine (mg/dl), mean ± SD | 1.1 ± 0.3 |

TABLE 2
Clinical Follow up (n = 8)

| | Base | | | | | | | | |
|---|---|---|---|---|---|---|---|---|---|
| | line | Week 1 | Week 2 | Week 3 | Week 4 | Week 5 | Week 6 | Week 7 | Week 8 |
| SBP sitting | 118 ± 21 | 109 ± 17 | 113 ± 16 | 111 ± 15 | 112 ± 16 | 116 ± 17 | 118 ± 20 | 121 ± 13 | 114 ± 14 |
| DBP sitting | 68 ± 7 | 67 ± 9 | 65 ± 8 | 65 ± 8 | 64 ± 5 | 67 ± 9 | 65 ± 4 | 70 ± 10 | 65 ± 10 |
| SBP supine | 118 ± 24 | 106 ± 15 | 110 ± 12 | 107 ± 14 | 111 ± 14 | 116 ± 17 | 114 ± 20 | 121 ± 13 | 120 ± 15 |
| DBP supine | 67 ± 9 | 62 ± 5 | 60 ± 9 | 60 ± 7 | 64 ± 8 | 69 ± 10 | 65 ± 6 | 72 ± 6 | 63 ± 6 |
| HR sitting | 66 ± 9 | 60 ± 9 | 61 ± 8 | 62 ± 8 | 65 ± 7 | 62 ± 6 | 61 ± 8 | 61 ± 4 | 62 ± 3 |
| HR supine | 62 ± 8 | 58 ± 5 | 63 ± 6 | 62 ± 11 | 64 ± 7 | 61 ± 7 | 61 ± 5 | 61 ± 6 | 62 ± 3 |
| Ghest pain | 0 | 0 | 0 | 0 | 0 | 0 | 1 | 0 | 1 |
| Hyper- | 0 | 0 | 0 | 0 | 0 | 0 | 0 | 0 | 0 |
| sensitivity | | | | | | | | | |
| Light- | 0 | 0 | 0 | 0 | 0 | 0 | 0 | 0 | 1 |
| headedness | | | | | | | | | |
| Cough | 0 | 0 | 0 | 0 | 0 | 0 | 2 | 0 | 1 |
| Ramipril | — | 2.5 × 1 | 2.5 × 1 | 2.5 × 2 | 2.5 × 2 | 5 × 2 | 5 × 2 | 7.5 × 2 | 7.5 × 2 |
| dose | | | | | | | | | |

TABLE 3
Lab Measures (n = 8)

| | Baseline | Week 1 | Week 3 | Week 5 | Week 7 |
|---|---|---|---|---|---|
| Potassium | 4.4 ± 0.5 | 4.3 ± 0.3 | 4.4 ± 0.4 | 4.3 ± 0.5 | 4.4 ± 0.5 |
| Creatinine | 1.1 ± 0.3 | 1.1 ± 0.3 | 1.2 ± 0.4 | 1.1 ± 0.3 | 1.1 ± 0.3 |
| Glucose | 105 ± 46 | 97 ± 29 | 96 ± 15 | 91 ± 18 | 91 ± 19 |

TABLE 4
Echo measures (n = 8)

| | Baseline | Week4 | Week8 |
|---|---|---|---|
| AS-Jet | 2.8 ± 0.2 | 2.8 ± 0.2 | 2.8 ± 0.3 |
| LVOT-velocity | 1.0 ± 0.2 | 1.1 ± 0.1 | 1.0 ± 0.2 |
| Cardiac output | 5.2 ± 1.1 | 5.9 ± 1.5 | 5.7 ± 1.4 |
| LVEF, % | 72 ± 4 | 73 ± 4 | 71 ± 7 |

The results of this study demonstrate, in contrast to the belief that ACE inhibitors are contra-indicated in the treatment of aortic valve disease, no adverse hemodynamic effects were seen with short-term ACE inhibitor treatment in n=8 patients with mild to moderate aortic stenosis, normal LV function and no clinical coronary artery disease.

### EXAMPLE 5

This example shows that treatment of patients to lower LDL levels through statin therapy correlates over time with lower calcification of the aortic valve which is an indication of a decrease in aortic valve disease progression as measured by Electron Beam Tomography (EBT) scanning.

Study population: Retrospective analysis were performed on 620 asymptomatic patients [513 men and 107 women, mean age 59 (range 30-86) years], referred by their primary, physicians for Electron Beam Tomography (EBT) scanning to evaluate coronary artery calcium, and who had undergone 2 consecutive EBT scans with an interscan interval of at least 6 months. Exclusion criteria were a history of left ventricular dysfunction or clinical evidence of coronary artery disease, including angina pectoris, previous coronary artery bypass graft surgery or previous percutaneous coronary interventions. Information on the presence or absence of traditional cardiovascular risk factors, including hypertension, family history or premature coronary artery disease, hyperlipidemia, smoking, diabetes mellitus and statin use, was obtained prior to the initial and follow-up EBT scans. Smoking was defined as the use of >10 cigarettes/day. Patients receiving insulin or oral hypoglycemic agents were classified as having diabetes mellitus. Patients were classified as having hypertension if they were receiving anti-hypertensive medications or had known but untreated hypertension. Hyperlipidemia was defined as use of cholesterol lowering medication or, in the absence of cholesterol lowering medication use, as having a total serum cholesterol >240 mg/dL. Patients were classified as receiving statin if they were receiving a statin drug at the time of both the initial and follow-up scans; no patients classified as not receiving statin were receiving statin at the time of either scan.

Scanning procedure: EBT scans were performed using an Imatron C-150XL Ultrafast Computed Tomographic Scanner (Imatron, South San Francisco, Calif.) with an acquisition time of 100 ms/image, ECG triggering at 80% of the RR interval and a slice thickness of 3 mm. A total of 30 consecutive images were obtained during two breath-holding periods from the aortic arch to the apex of the heart. Foci with a density of >130 Houndsfield units and area ≧3 contiguous pixels were regarded as calcification. Calcium scores for the coronary arteries and the aortic valve were quantified by the method of Agatston et al (J.Am.Coll.Cardiol.1990;15(4):827-32) and by the calcium volumetric score determined by the method of isotropic interpolation (Callisster et al, Radiology 1998; 208(3):807-14). The aortic valve was identified as the structure between the left ventricular cavity and the ascending aorta and was usually present in 3 to 4 consecutive images. Aortic valve leaflet calcium was defined as present if calcium was seen in the continuous plane between the left ventricular cavity and the ascending aorta. Calcium within the aortic sinuses and aortic wall were excluded from analysis.

Statistical analyses: Rates of change of the Agatson (J.Am.Coll.Cardiol.1990; 15(4):827-32) and volumetric (Callisster et al, Radiology 1998; 208(3):807-14) scores for aortic valve calcium (AVC) were expressed as % change per year by the formula: [(AVC on follow-up EBT Scan−AVC on initial EBT Scan)/(AVC on initial EBT Scan×Interscan Interval in years)]×100. Definite progression of AVC was defined as volumetric or Agatston score change of ≧18%, which is 2 times the published median interscan variability for the volumetric score (8.9%) by the formula: [(AVC on follow-up EBT Scan−AVC on initial EBT Scan)/(AVC on initial EBT Scan×Interscan Interval in years)]×100. Definite progression of AVC was defined as volumetric or Agatston score change of ≧18%, which is 2 times the published median interscan variability for the volumetric score (8.9%) by the formula: [(AVC on follow-up EBT Scan−AVC on initial EBT Scan)/(AVC on initial EBT Scan×Interscan Interval in years)]×100. Definite progression of AVC was defined as volumetric or Agatston score change of ≧18%, which is 2 times the published median interscan variability for the volumetric score (8.9%) (Callisster et al, Radiology, 1998). The frequency of definite AVC progression between those receiving statin therapy and those not receiving statin therapy was compared by chi-square test. Clinical characteristics of patients receiving and not receiving statin therapy were compared using t-test for continuous variables and chi-square test for categorical variables. Multivariate analysis was performed to evaluate the relationship between increasing AVC score and cardiovascular risk factors and statin therapy. Significance was defined as p<0.05.

Characteristics of patients with and without statin therapy: Of the 65 patients with an AVC volumetric score >10 on the initial scan, 28 (43%) were receiving statin therapy at the time of both the initial and follow-up scans. None of the remaining 37 patients were receiving statins at the time of either the initial or follow-up scans. Patients receiving statins were twice as likely to carry a diagnosis of hyperlipidemia (100% vs. 46%, p=0.02) and twice as likely to carry a diagnosis of hypertension (50% vs. 24%, p=0.03) as patients not receiving statin therapy. However, patients not receiving statin therapy and those receiving statin therapy were of similar age, had similar prevalence of diabetes, smoking and family history of coronary artery disease, and had similar interscan intervals.

Association of statin therapy with lower AVC accumulation rates: Statin therapy was associated with a highly significant lower unadjusted rate of AVC accumulation, as assessed by % change in both AVC volumetric score (p=0.009) and AVC Agatston score (p=0.005). Specifically, rates of change for AVC volumetric score were 41.2±47.9%/year (range −25 to +197%/year) for patients not receiving statin therapy. vs. 14.9±39.4%/year (mean±SD, range −35 to +182%/year) for patients on statin therapy. Rates of change for AVC Agatston score were 67.7±100.8%/year (range −21.5 to +439.5%/year) for patients not receiving statin therapy vs. 19.2±42.2%/year (mean±SD, range −26.8 to +201.5%/year) for patients receiving statin therapy.

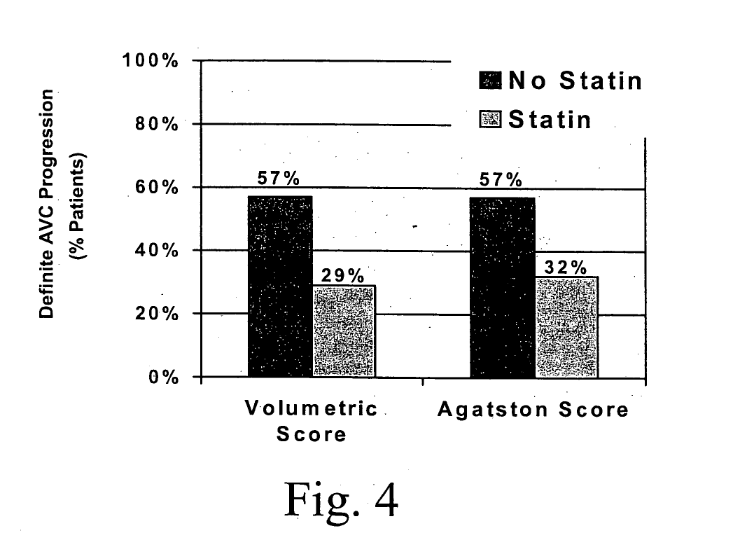

As shown in Table 5, there was a significant inverse association between statin therapy and rate of AVC accumulation (correlation coefficient=−33,p=0.02). Similar trends were seen using the Agatston score. In contrast, definite AVC progression, as determined by the volumetric score, was seen in 21 patients (57%) not receiving statin therapy vs. 8 patients (29%) receiving statin therapy (p<0.05) (see FIG. 4).

TABLE 5
Correlation of traditional coronary risk factors and statin use
with increased AVC accumulation.

| | Correlation | P |
|---|---|---|
| Variables | Coefficient | value |
| By Volumetric Score: | | |
| Family history of CAD | −2.4 | 0.84 |
| Hypertension | −2.1 | 0.86 |
| Smoking | −1.5 | 0.94 |
| Hyperlipidemia | 15.2 | 0.31 |
| Diabetes Mellitus | 44.0 | 0.01 |
| Statin Use | −32.6 | 0.02 |
| By Agatston Score: | | |
| Family history of CAD | 6.5 | 0.76 |
| Hypertension | −22.6 | 0.30 |
| Smoking | 21.9 | 0.53 |
| Hyperlipidemia | 22.9 | 0.40 |
| Diabetes Mellitus | 79.9 | 0.01 |
| Statin Use | −54.4 | 0.03 |

These results demonstrate that there is a strong correlation between statin use and a lower rate of aortic valve calcium accumulation and suggests that this pharmacological treatment is useful to inhibit aortic valve disease progression. This study also demonstrates the utility of EBT in quantifying AVC change in aortic valve disease over time.

The embodiments of the invention in which an exclusive property or privilege is claimed are defined as follows:

1. A method of decreasing the amount and/or biological activity of angiotensin II in an aortic valve in an animal, comprising administering to the animal an amount of an angiotensin II inhibiting agent, selected from the group consisting of angiotensin converting enzyme antagonists and angiotensin II type 1 receptor antagonists, effective to decrease the amount and/or biological activity of angiotensin II in the aortic valve in the animal.

2. The method of claim 1 wherein the angiotensin II inhibiting agent is an antagonist of angiotensin converting enzyme.

3. The method of claim 2 wherein the angiotensin converting enzyme antagonist is a pharmacological agent selected from the group consisting of a pharmaceutical compound that inhibits angiotensin converting enzyme, an antisense angiotensin converting enzyme nucleic acid molecule, an anti-angiotensin converting enzyme antibody, an angiotensin converting enzyme blocking peptide and an angiotensin converting enzyme ribozyme.

4. The method of claim 3 wherein said angiotensin converting enzyme antagonist is selected from the group consisting of ramipril, quinapril, captopril, lisinopril, benazepril, enalapril and fosinopril.

5. The method of claim 2 wherein said angiotensin converting enzyme antagonist is effective to decrease the amount and/or biological activity of angiotensin converting enzyme carried into the aortic valve by low density lipoproteins.

6. The method of claim 5 wherein said angiotensin converting enzyme antagonist is a pharmaceutical compound that decreases plasma low density lipoprotein levels in the animal.

7. The method of claim 6 wherein said pharmaceutical compound is selected from the group consisting of statin, lovastatin, pravastatin, simvastatin, atorvastatin, rosuvastatin; nicotinic acid, bile acid binding resins, fibric acid derivatives and cholesterol adsorption inhibitors.

8. The method of claim 5 wherein said angiotensin converting enzyme antagonist is a molecule that inhibits binding and/or retention of angiotensin converting enzyme-containing low density lipoprotein particles in an aortic valve lesion.

9. The method of claim 1 wherein a angiotensin II type 1 receptor antagonist is introduced into the animal.

10. The method of claim 9 wherein said angiotensin II type 1 receptor antagonist is a pharmacological agent selected from the group consisting of a pharmaceutical compound, an antisense angiotensin II type 1 receptor nucleic acid molecule, an anti-angiotensin II type 1 receptor antibody, an angiotensin II type 1 receptor blocking peptide and an angiotensin II type 1 receptor ribozyme.

11. The method of claim 10 wherein said pharmaceutical compound is selected from the group consisting of losartan, valsartan, irbesatan, telmesartan and candesartan.

12. The method of claim 1 wherein the animal is exhibiting aortic valve disease, and the amount of the introduced agent is effective to prevent progression and/or complications of aortic valve disease.

13. The method of claim 12 wherein the aortic valve disease is aortic sclerosis.

14. The method of claim 12 wherein the aortic valve disease is aortic stenosis.

15. The method of claim 1 wherein the agent is introduced into the animal by a method selected from the group consisting of injection, transdermal application and as a component of a lipid complex.

16. The method of claim 15 further comprising a plurality of angiotensin II antagonist molecules, specific for the inhibition of angiotensin converting enzyme and/or angiotensin II type 1 receptor that are introduced into the animal.
